(19) United States
(12) Patent Application Publication
(10) Pub. No.: US 2023/0277490 A1
(43) Pub. Date: Sep. 7, 2023

(54) GLUTAMINE AS AN ANTICANCER THERAPY IN SOLID TUMORS

(71) Applicant: CEDARS-SINAI MEDICAL CENTER, Los Angeles, CA (US)

(72) Inventors: Jun Gong, Sherman Oaks, CA (US); Andrew Hendifar, Santa Monica, CA (US); Neil Bhowmick, Beverly Hills, CA (US); Hayato Muranaka, Los Angeles, CA (US)

(73) Assignee: CEDARS-SINAI MEDICAL CENTER, Los Angeles, CA (US)

(21) Appl. No.: 18/016,768

(22) PCT Filed: Jul. 30, 2021
(86) PCT No.: PCT/US21/43961

§ 371 Date: Jan. 18, 2023

Related U.S. Application Data
(60) Provisional application No. 63/059,726, filed on Jul. 31, 2020.

(51) Int. Cl. A61K 31/198; A61K 31/7068; A61K 31/337; A61P 35/00; A61P 1/18

(52) U.S. Cl. CPC A61K 31/198; A61K 31/7068; A61K 31/337; A61P 35/00; A61P 1/18

(57) ABSTRACT

The present invention provides for methods of treating solid tumors, particularly, KRas mutated solid tumors and KRas mutated pancreatic cancer. The method includes administering glutamine alone or in combination with an anti-cancer agent; for example, gemcitabine or nab-paclitaxel. The method includes administering glutamine and radiotherapy, or glutamine and immunotherapy to treat these cancers.

### CROSS-REFERENCE TO RELATED APPLICATIONS

This application includes a claim of priority under 35 U.S.C. §119(e) to U.S. provisional patent application No. 63/059,726, filed Jul. 31, 2020, the entirety of which is hereby incorporated by reference.

### FIELD OF INVENTION

This invention relates to the treatment of solid tumors.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Cytotoxic chemotherapy remains the preferred treatment for advanced or unresectable pancreatic cancer with combination regimens including 5-fluorouracil (5-FU), leucovorin, irinotecan, and oxaliplatin (FOLFIRINOX) or gemcitabine and nab-paclitaxel having recently been established as first-line standards in patients with good performance status (PS). For those with poor PS, gemcitabine alone has remained a standard cytotoxic agent in the treatment of metastatic pancreatic cancer. The administration rates of second-line and third-line chemotherapy in metastatic pancreatic cancer has approximated 45% and 21%, respectively, following failure of gemcitabine. These values reinforce the importance of optimizing first-line chemotherapy given that the aggressive nature of advanced pancreatic cancer is underscored by progressive therapeutic resistance and a dismal 5-year survival rate of 3%. Accordingly, there remains an urgent need for additional treatment options for these cancers, as well as other solid tumors.

### SUMMARY OF THE INVENTION

The following embodiments and aspects thereof are described and illustrated in conjunction with compositions and methods which are meant to be exemplary and illustrative, not limiting in scope.

Various embodiments provide for a method of treating a solid tumor in a subject in need thereof, comprising: administering a therapeutically effective amount of glutamine to the subject.

In various embodiments, the method can further comprise administering one or more treatment cycles of a therapeutically effective amount of anticancer agent, an immunotherapeutic agent, or radiation therapy to the subject.

In various embodiments, the method can comprise administering the glutamine about 3 days to 2 weeks prior to initiating the first treatment cycle of the anticancer agent, the immunotherapeutic agent, or radiation therapy.

In various embodiments, the method can comprise administering the glutamine about 1 week prior to initiating the first treatment cycle of the anticancer agent, the immunotherapeutic agent, or radiation therapy.

In various embodiments, the first treatment cycle of the anticancer agent or the immunotherapeutic agent can comprise administering the anticancer therapy or the immunotherapeutic agent on about days 1, 8, and 15, or wherein the first treatment cycle of the radiation therapy can comprise administering the radiation therapy once, twice or three times a week for about four weeks. In various embodiments, the first treatment cycle can be about 28 days.

In various embodiments, the glutamine can be L-glutamine. In various embodiments, the glutamine can be D-glutamine.

In various embodiments, administering the glutamine can comprise administering about 0.1 g/kg-1.2 g/kg of glutamine per day. In various embodiments, administering the glutamine can comprise administering about 0.2 g/kg-0.6 g/kg of glutamine per day.

In various embodiments, administering the glutamine can comprise administering glutamine twice daily. In various embodiments, administering the glutamine can comprise 10 g or 15g of glutamine twice daily. In various embodiments, administering the glutamine can comprise administering an amount of glutamine to achieve about 10 mM to 50 mM concentration of glutamine in the subject. In various embodiments, administering the glutamine can comprise administering an amount of glutamine to achieve at about 5 to 50 times the physiologic concentration of glutamine in the subject.

In various embodiments, the solid tumor can be a KRas mutated cancer. In various embodiments, the solid tumor can be pancreatic cancer. various embodiments, the solid tumor can be KRas mutated pancreatic cancer. In various embodiments, the anticancer agent can be chemotherapy. In various embodiments, the chemotherapy can be gemcitabine. In various embodiments, the chemotherapy can be nab-paclitaxel or paclitaxel.

Various embodiments provide for a method of treating a solid tumor in a subject in need thereof, comprising: administering a therapeutically effective amount of glutamine to the subject prior to administering one or more treatment cycles of a therapeutically effective amount of anticancer agent, an immunotherapeutic agent, or radiation therapy to the subject; continuing to administer the therapeutically effective amount of glutamine to the subject; and administering one or more treatment cycles of a therapeutically effective amount of anticancer agent, an immunotherapeutic agent, or radiation therapy to the subject.

In various embodiments, the glutamine can be L-glutamine. In various embodiments, the glutamine can be D-glutamine.

In various embodiments, the method can comprise administering the therapeutically effective amount of glutamine to the subject about 3 days to 2 weeks prior to administering one or more treatment cycles of the therapeutically effective amount of anticancer agent, an immunotherapeutic agent, or radiation therapy to the subject. In various embodiments, the method can comprise administering the therapeutically effective amount of glutamine to the subject about 1 week prior to administering one or more treatment cycles of the therapeutically effective amount of anticancer agent, an immunotherapeutic agent, or radiation therapy to the subject.

In various embodiments, the therapeutically effective amount of glutamine can be about 0.1g/kg-1.2 g/kg per day. In various embodiments, the therapeutically effective amount of glutamine can be about 0.2g/kg -0.6 g/kg per day.

In various embodiments, the one or more treatment cycles of the therapeutically effective amount of anticancer agent can comprise administering one or more treatment cycles of gemcitabine. In various embodiments, each treatment cycle of gemcitabine can comprise about 600 mg/m²-1000 mg/m² of gemcitabine administered on day 1, day 8 and day 15.

In various embodiments, the one or more treatment cycles of the therapeutically effective amount of anticancer agent can comprise administering one or more treatment cycles of Nab-paclitaxel. In various embodiments, each treatment cycle of Nab-paclitaxel can comprise about 75 mg/m²-125 mg/m² of Nab-paclitaxel administered on day 1, day 8 and day 15.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows relative cell numbers on samples treated with glutamine and varying concentrations of gemcitabine. The combination treatment of elevated concentrations of glutamine and gemcitabine resulted in a dramatic increase in cell death compared to either gemcitabine or glutamine alone.

FIG. 2 shows basal metabolic rate of pancreatic cancer cells are elevated by gemcitabine and further elevated still by the combination of 40 mM glutamine and 0.5 μM gemcitabine

FIG. 3 shows a heat map of the differential metabolite accumulation with glutamine and gemcitabine treatment, as single agent and in combination.

FIG. 4 shows PDAC cells treated with glutamine supplementation had enhancement of PDAC cell death with increasing concentrations of gemcitabine.

FIG. 5 shows glutamine supplementation decreased cancer cell viability with increasing concentrations of gemcitabine even in the face of glutaminase inhibition.

FIG. 6 shows sequential cell counting for various concentrations of glutamine and gemcitabine.

FIG. 7 shows the effects of L-Gln supplementation on PDAC cells. When CB-839 was used in vitro in Mia PaCa-2 cells, we saw some antitumor efficacy (top graph, 3^{rd} bar), but notably we saw greater antitumor activity with high-dose L-Gln supplementation (top graph, 2^{nd} bar). Of note, the antitumor activity of L-gln occurred independently of glutaminase inhibition with CB-839 (top graph, 4^{th} bar). On the bottom graph, we saw over time, 40 mM L-Gln was consistently better with antitumor efficacy in PDAC cells in vitro compared to normal, physiologic (control) 2 mM L-Gln

FIG. 8 shows the effects of high-dose L-Gln on TCA cycle. We compared the metabolite levels through the TCA cycle but noticed that 40 mM L-Gln despite having higher Gln and glutamate concentrations, did not affect the majority of TCA cycle metabolites—suggesting that its anticancer mechanism involved a different pathway.

FIG. 9 shows the effects of high-dose L-Gln on nucleotides. 40 mM L-glutamine significantly depleted intracellular nucleotide stores compared to normal, physiologic (control) L-Gln at 2 mM.

FIG. 10 shows that L-Glutamine Supplementation Enhances the Effect of Gemcitabine in PDAC Cells. The ability of L-Gln supplementation to deprive intracellular nucleotide stores makes it an attractive agent to pair with gemcitabine, a standard chemotherapy used in the treatment of PDAC patients whose activity is also based on its ability to inhibit DNA synthesis. Indeed, L-Gln 40 mM consistently enhanced the antitumor effects of gemcitabine across several PDAC cell lines, when compared to 2 mM L-Gln.

FIG. 11 shows effects of high-dose L-Gln on amino acids. 40 mM L-Gln significantly depleted intracellular essential and conditionally amino acids compared to 2 mM L-Gln in PDAC cells in vitro.

FIG. 12 shows effects of high-dose L-Gln on intracellular Na⁺ in MIA PaCa-2 cells. 40 mM L-Gln increases intracellular sodium levels as measured by sodium green fluorescence intensity in MIA PaCa-2 cells in vitro.

FIG. 13 shows the effect of Na, K-ATPase inhibition on cell growth in MIA PaCa-2 cells. A Na/K ATPase inhibitor, Ouabain, was added into PDAC cells in vitro, a similar effect was elicited as Ouabain leads to build up of intracellular sodium similar to 40 mM L-Gln—here we illustrate a similar principle to L-Gln, that with Ouabain if you increase intracellular sodium (left), you result in greater PDAC cell death (right).

FIG. 14 shows the effects of D-Gln on PDAC cell growth. Similar anticancer effects were demonstrated with high dose D-Gln as well in PDAC cells in vitro, the stereoisomer of L-Gln.

FIG. 15 shows the effects of high-dose D-Gln on intracellular Na+ in MIA PaCa-2 cells. Similar effects on intracellular sodium influx with high dose D-Gln were demonstrated as well in PDAC cells in vitro.

FIG. 16 shows the effects of high-dose L-Gln, D-Gln and L-alanyl-L-glutamine on colony formation. Similar anticancer effects were demonstrated with high dose L-Gln and D-Gln in PDAC cells in vitro.

FIG. 17 shows the effects of high-dose L-Gln and gemcitabine on colony formation (lower effective doses of chemotherapy). As L-Gln supplementation enhances the efficacy of chemotherapy, we tested whether L-Gln can lower the effective dose of gemcitabine. We show that 20 mM L-Gln can enhance the efficacy gemcitabine.

FIG. 18 shows the effects of high-dose L-Gln and 5-FU on colony formation (lower effective doses of chemotherapy). We show in circles 30 mM L-Gln and a lower concentration of 5-FU chemo (another standard chemo drug in PDAC) at 0.01 uM is as effective or even more effective than 5-FU at 0.1 uM.

FIG. 19 shows the effects of high-dose L-Gln and paclitaxel on colony formation. L-Gln also enhances the efficacy of paclitaxel (another standard chemotherapy drug in PDAC).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. In some embodiments, the subject is a human.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be including within the scope of this term. In some embodiments, the mammal is a human.

“Therapeutically effective amount” as used herein refers to that amount which is capable of achieving beneficial results in a patient having cancer. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or lessen the disease even if the treatment is ultimately unsuccessful.

“L-glutamine” as used herein refers to the L-enantiomer of glutamine wherein at least 51% of a quantity of glutamine is L-glutamine. In various embodiments, at least 60% of glutamine is L-glutamine. In various embodiments, at least 75% of glutamine is L-glutamine. In various embodiments, at least 80% of glutamine is L-glutamine. In various embodiments, at least 90% of glutamine is L-glutamine. In various embodiments, at least 95% of glutamine is L-glutamine. In various embodiments, at least 96% of glutamine is L-glutamine. In various embodiments, at least 97% of glutamine is L-glutamine. In various embodiments, at least 98% of glutamine is L-glutamine. In various embodiments, at least 99% of glutamine is L-glutamine. In various embodiments, at least 99.5% of glutamine is L-glutamine.

“D-glutamine” as used herein refers to the D-enantiomer of glutamine wherein at least 51% of a quantity of glutamine is D-glutamine. In various embodiments, at least 60% of glutamine is D-glutamine. In various embodiments, at least 75% of glutamine is D-glutamine. In various embodiments, at least 80% of glutamine is D-glutamine. In various embodiments, at least 90% of glutamine is D-glutamine. In various embodiments, at least 95% of glutamine is D-glutamine. In various embodiments, at least 96% of glutamine is D-glutamine. In various embodiments, at least 97% of glutamine is D-glutamine. In various embodiments, at least 98% of glutamine is D-glutamine. In various embodiments, at least 99% of glutamine is D-glutamine. In various embodiments, at least 99.5% of glutamine is D-glutamine.

For the past 10 years, starving or depriving pancreatic cancer (e.g., PDAC) of glutamine has been a principal strategy. However, efforts to antagonize glutamine metabolism with CB-839, for example, an inhibitor of glutaminase has failed in PDAC mouse models.

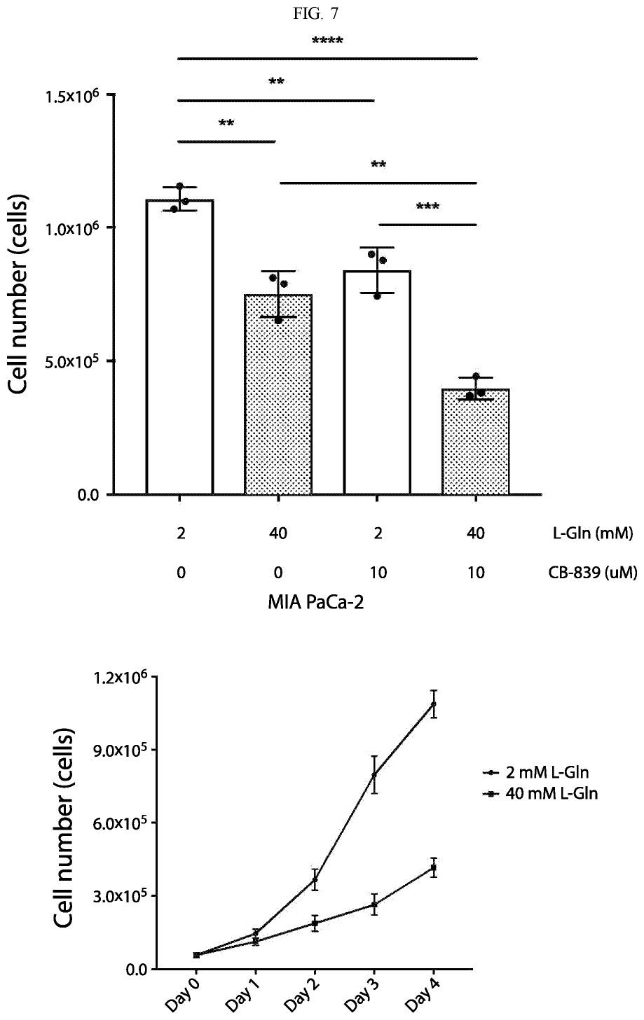

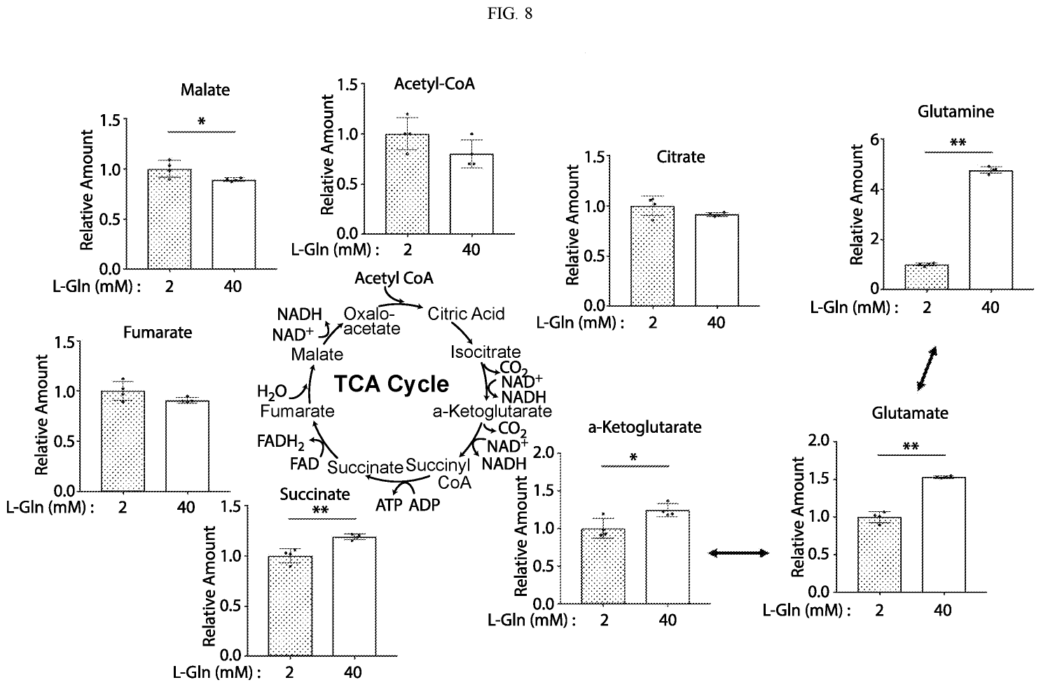

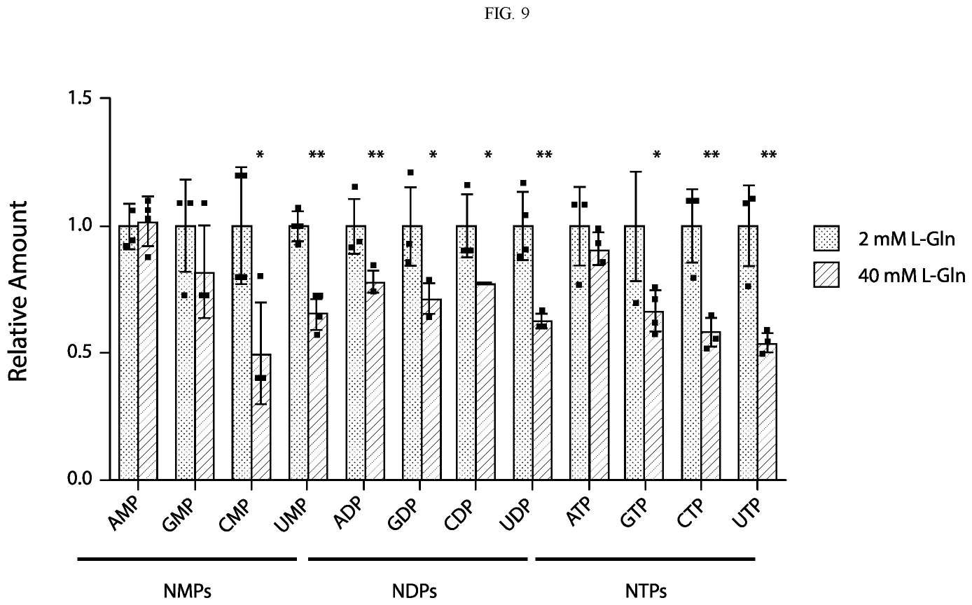

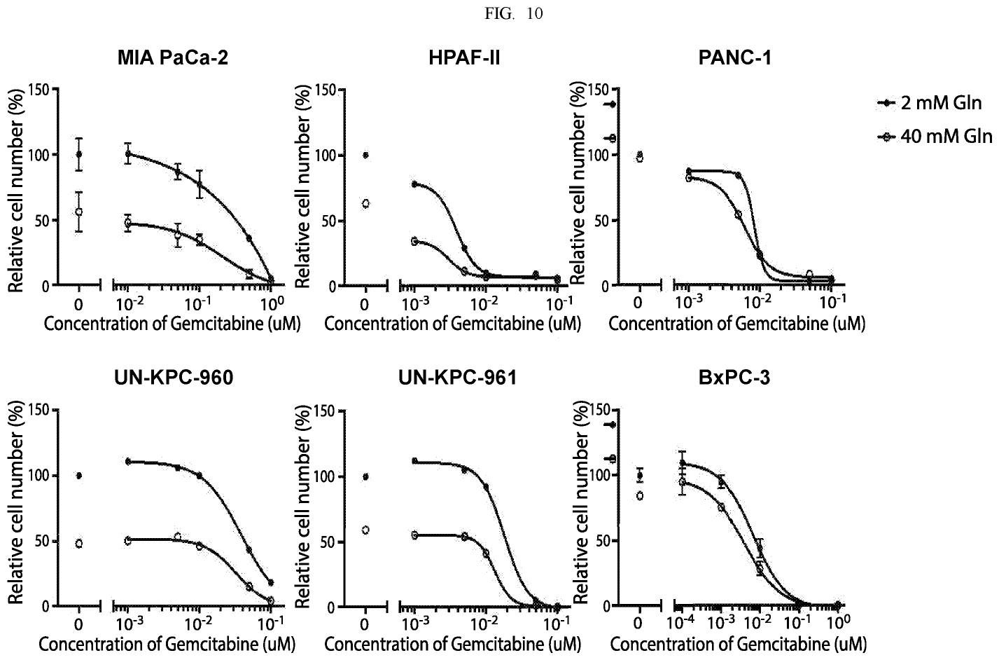

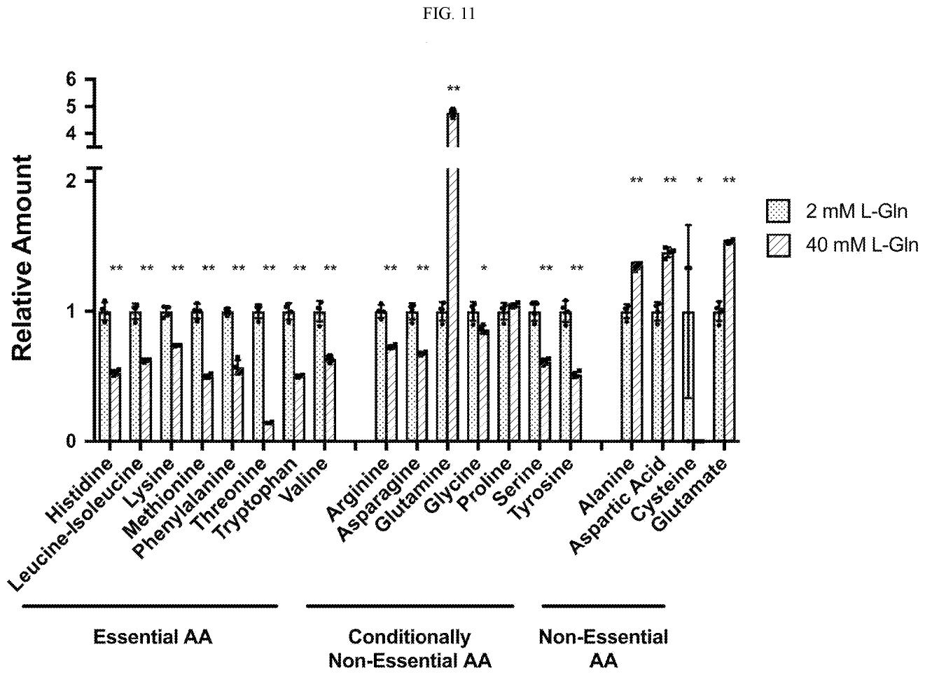

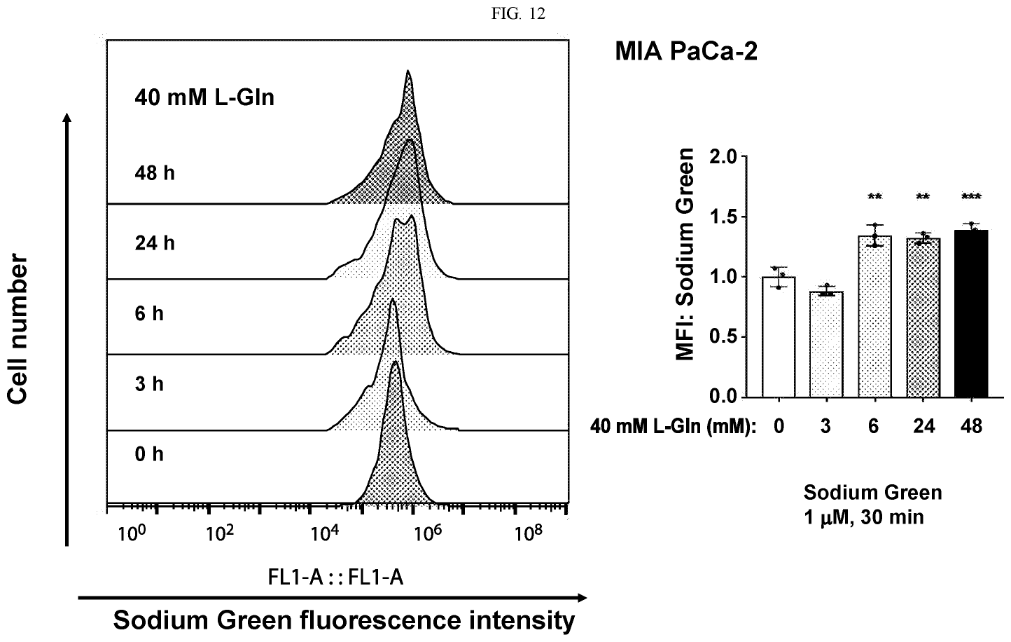

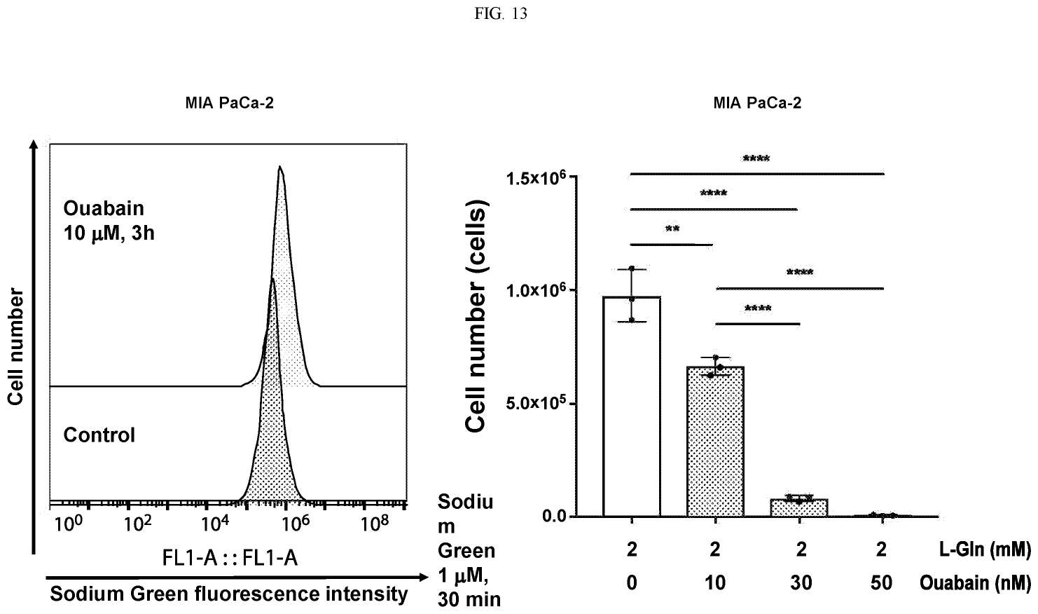

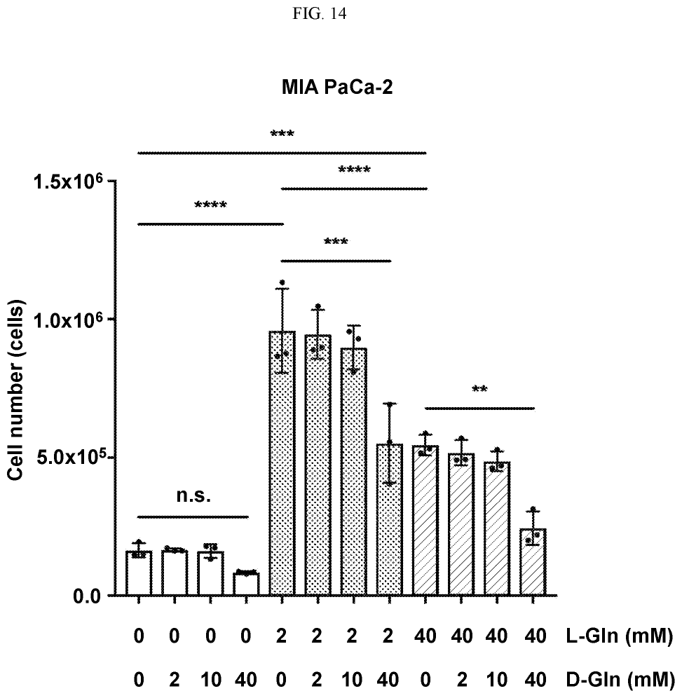

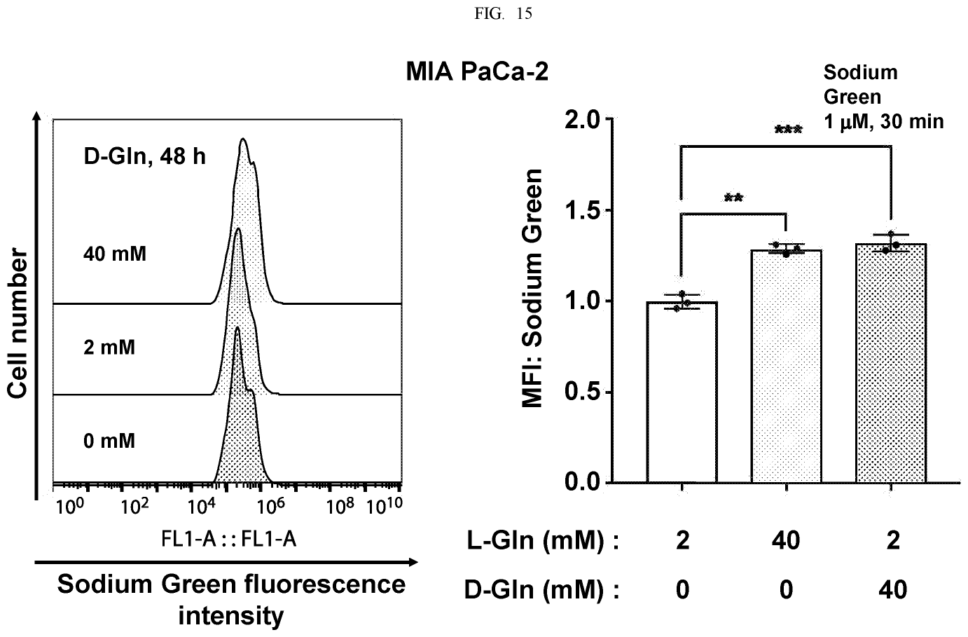

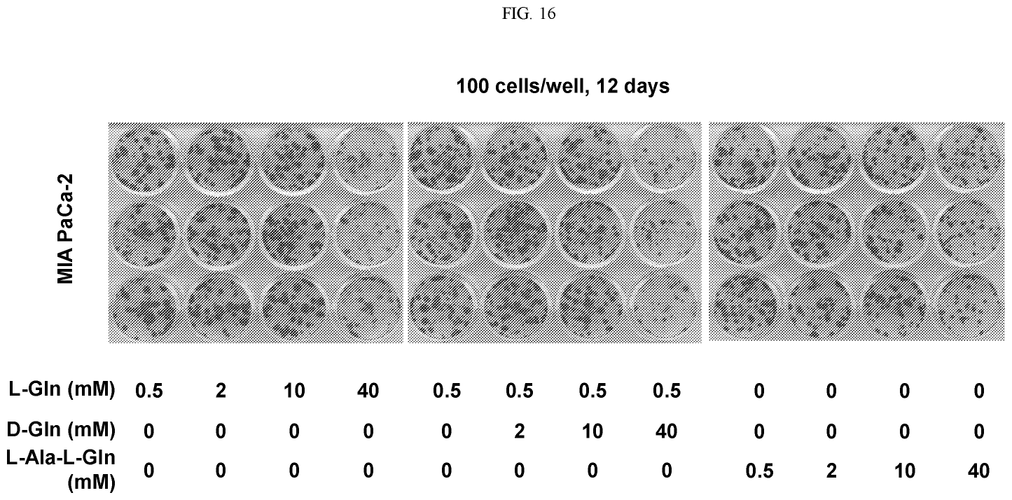

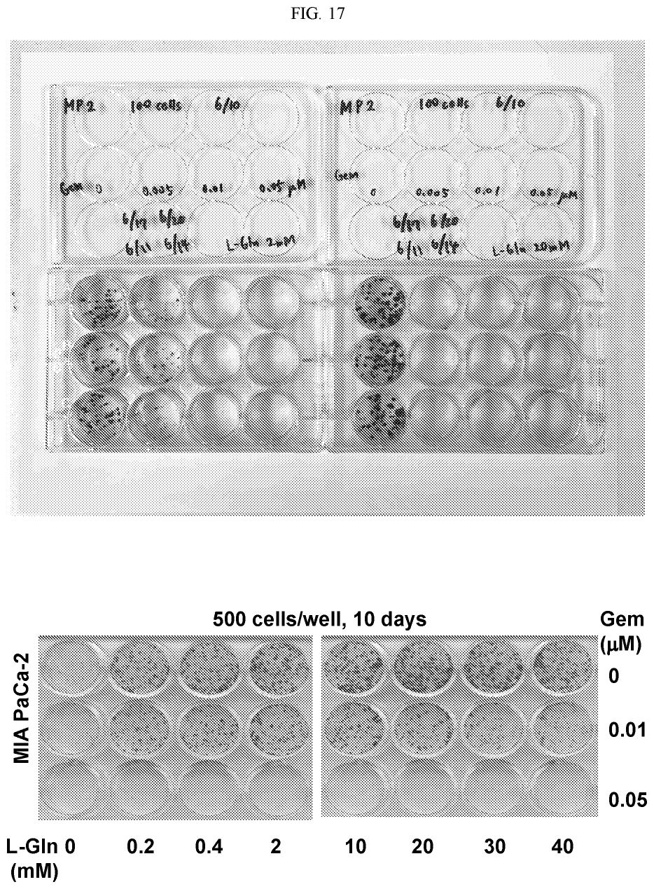

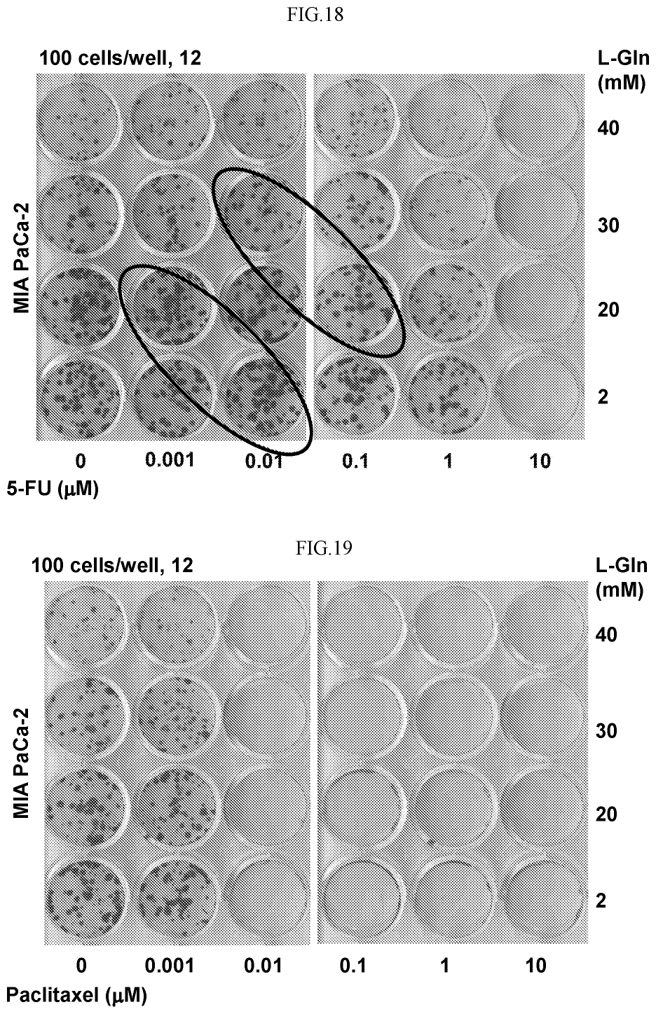

Described herein, we used CB-839 in vitro in Mia PaCa-2 cells, and we saw some antitumor efficacy (FIG. 7, 3rd bar), but notably we saw greater antitumor activity with high-dose L-Gln supplementation (FIG. 7, 2nd bar). Of note, the antitumor activity of L-gln occurred independently of glutaminase inhibition with CB-839 (FIG. 7, 4th bar). On FIG. 7, we saw over time, 40 mM L-Gln was consistently better with antitumor efficacy in PDAC cells in vitro compared to normal, physiologic (control) 2 mM L-Gln.

We show herein that glutamine deprivation through glutaminase inhibition increases cancer cell survival and potentiates resistance to gemcitabine in PDAC cells. Instead, glutamine supplementation enhances sensitivity to gemcitabine in PDAC cells, even in the face of glutaminase inhibition.

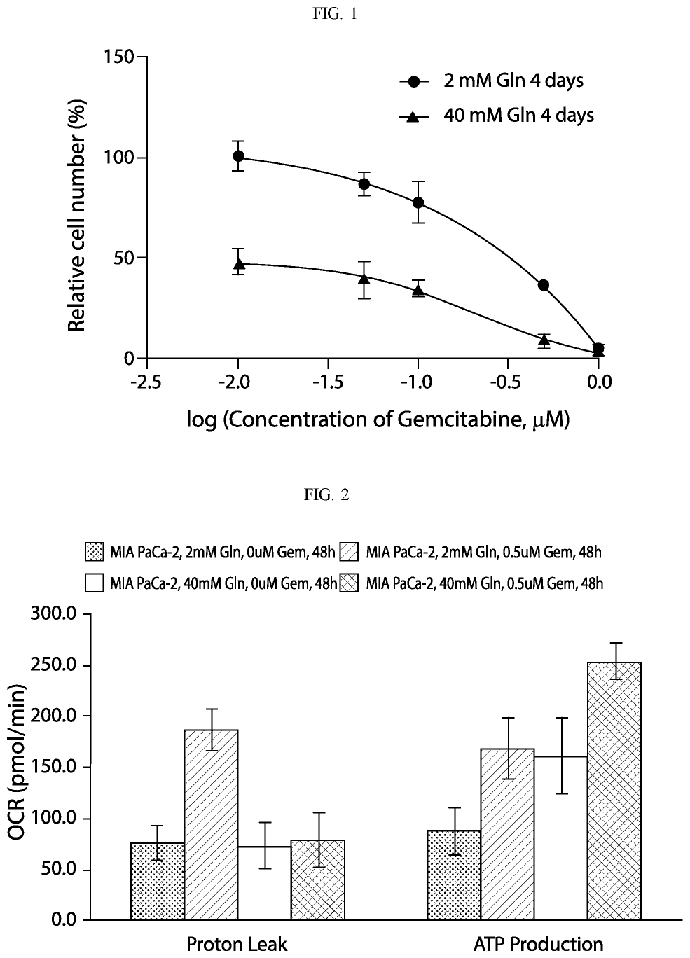

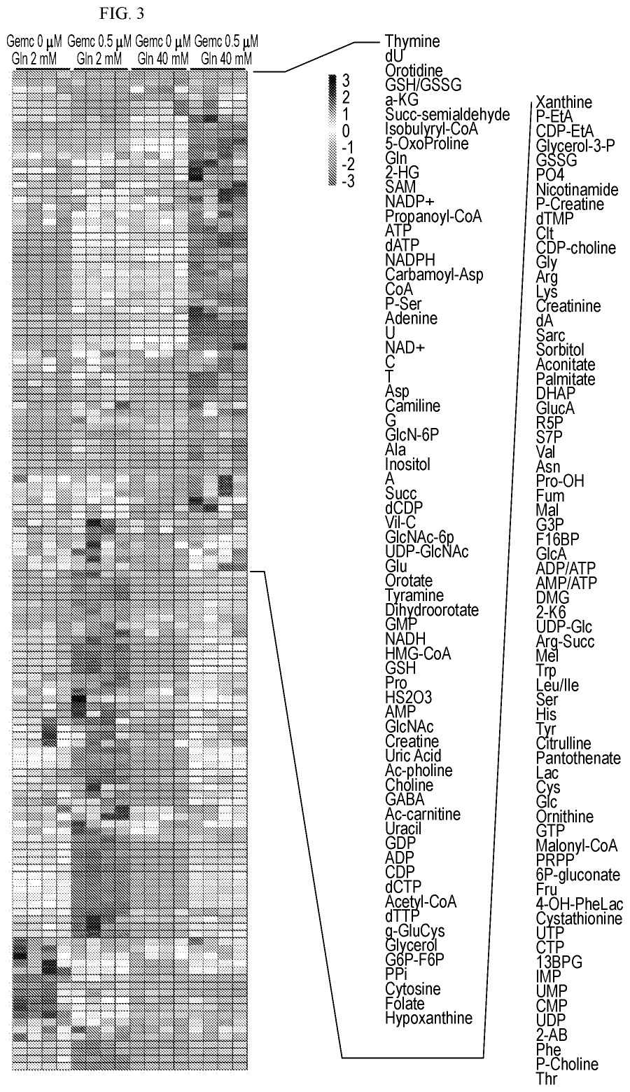

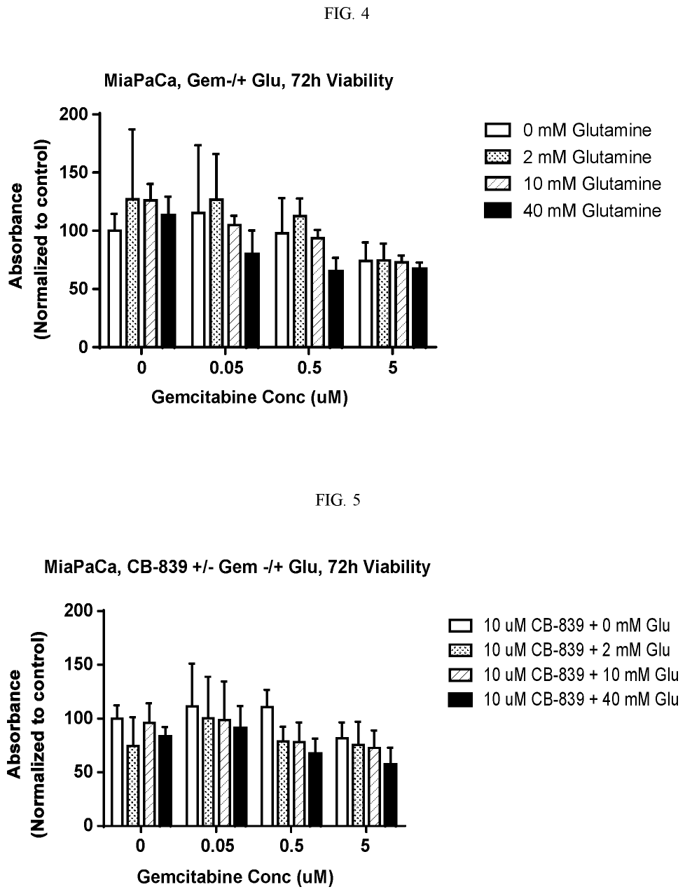

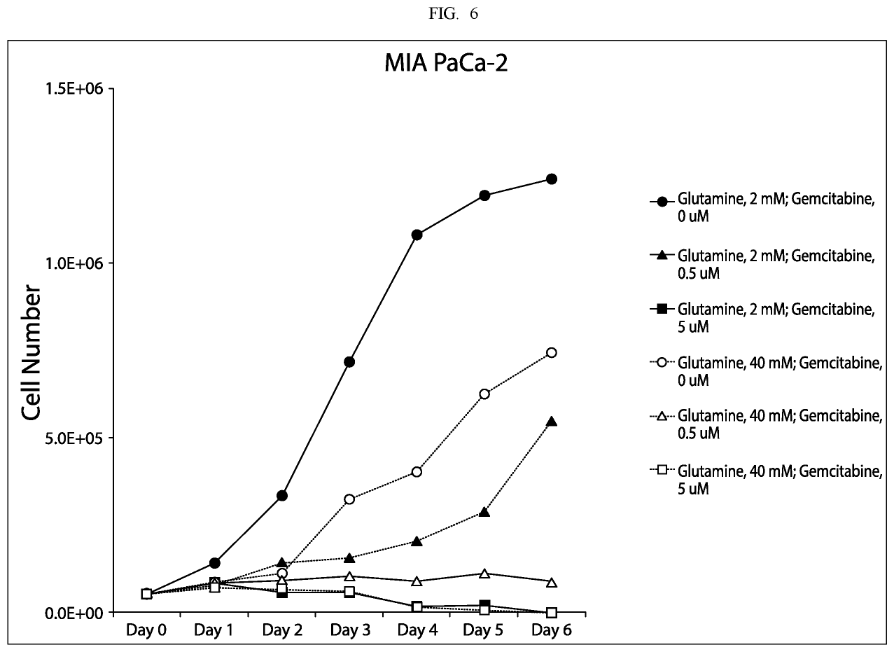

Pancreatic cancer cells (MIAPaCa human cell line) were found to develop resistance to gemcitabine. We tested how elevated glutamine affects gemcitabine efficacy. To our surprise, the combination treatment of elevated concentrations of glutamine and gemcitabine resulted in a dramatic increase in cell death compared to either gemcitabine or glutamine alone (FIG. 1). A dose response of gemcitabine in the presence of either 2 mM glutamine (physiologic concentration) or 40 mM glutamine (super-physiologic concentration) demonstrated that 40 mM glutamine alone could reduce total cell number after 4 days of treatment by half. The addition of gemcitabine to 40 mM glutamine reduced IC50 from 0.6661 μM gemcitabine to 0.1794 μM gemcitabine. We found that the basal metabolic rate of pancreatic cancer cells to be elevated by gemcitabine and further elevated still by the combination of 40 mM glutamine and 0.5 μM gemcitabine. While it was not a surprise that high glutamine concentrations may increase cellular metabolic rate, it did not explain the extraordinary induction of cell death potentiated by the combination treatment (FIG. 2). Interestingly, mitochondrial proton leakage induced by gemcitabine was reduced by additional glutamine treatment. We next tested the metabolic impact of super-physiologic glutamine concentration (40 mM) on the gemcitabine response (0.5 μM) of pancreatic cancer cells through standard metabolomic mass spectrometric analysis. The heat map in FIG. 3 illustrates the differential metabolite accumulation with glutamine and gemcitabine treatment, as single agent and in combination. There were a number of metabolic changes that were only observed with the combination treatment. We found that the combination treatment caused an accumulation of free nucleotides, presumably available for DNA damage repair. However, there was greater DNA damage accumulation in the combination treatment arm over either of the single agents. The consequential metabolic changes appeared to be the interesting accumulation of vitamin C and S-adenosine methionine (SAM). Both vitamin C and SAM at high concentrations were found to be consequential to the elevated cell death observed with high glutamine and gemcitabine. Importantly, high glutamine administration would reduce the concentration of gemcitabine required to cause significant cancer cell death.

Embodiments of the present invention are based, at least in part, on these finding.

Various embodiments the present invention provide for a method of treating a solid tumor in a subject in need thereof, comprising: administering a therapeutically effective amount of glutamine to the subject. In various embodiments, the glutamine is D-glutamine. The use of D-glutamine can be beneficial as it can limit the metabolism of glutamine for the generation of ATP. In various embodiments, the glutamine is L-glutamine.

In various embodiments, the method further comprises administering one or more treatment cycles of a therapeutically effective amount of anticancer agent to the subject. In various embodiments, the method further comprises administering one or more treatment cycles of a therapeutically effective amount of an immunotherapeutic agent to the subject. In various embodiments, the method further comprises administering one or more treatment cycles of a therapeutically effective amount of radiation therapy to the subject.

In various embodiments, there is one treatment cycle. In various embodiments, there are 2, 3, 4, or 5 treatment cycles. In various embodiments, there are 6, 7, 8, 9 or 10 treatment cycles. In still other embodiments, there are 11, 12, 13, 14, 15, 16, 17, 18 19 or 20 treatment cycles. In various embodiments, there may be a break between each treatment cycle; for example, a 3, 5 or 7-day break between each treatment cycle, or a 1, 2, 3, or 4 week break between each treatment cycle.

In various embodiments, the method comprises administering the glutamine about 1 week prior to initiating the first treatment cycle of the anticancer agent. In various embodiments, the method comprises administering the glutamine about 1 week prior to initiating the first treatment cycle of the immunotherapeutic agent. In various embodiments, the method comprises administering the glutamine about 1 week prior to initiating the first treatment cycle of the radiation therapy. In various embodiments, administering the glutamine comprises administering glutamine twice daily. In various embodiments, administering the glutamine comprises administering about 10, 20, 30, 40, 50, or 60 g/day. In various embodiments, administering the glutamine comprises administering about 12, 24, 36, 48, 60, or 72 g/day. In various embodiments, administering the glutamine comprises administering about 12 g/day. In various embodiments, administering the glutamine comprises administering about 24 g/day. In various embodiments, administering the glutamine comprises administering about 36 g/day. In various embodiments the dosage can be administered once daily, twice daily or three times daily. For example, if the dosage is about 12 g/day, administered twice a day, then about 6 g is administered each time. In particular embodiments, administering the glutamine comprises administering 10 g or 15 g of glutamine twice daily. In particular embodiments, administering the glutamine comprises administering about 6 g, 12 g, or 18 g of glutamine twice daily. The administration of glutamine can continue through the treatment cycle with the anticancer agent, the immunotherapeutic agent, or the radiation therapy.

In various embodiments, administering the glutamine depends on the weight of the subject. Thus in some embodiments administering the glutamine comprises administering about 0.1-0.3 g/kg per day. In some embodiments, administering the glutamine comprises administering about 0.05-0.15 g/kg per day. In some embodiments, administering the glutamine comprises administering about 0.2-0.6 g/kg per day. In some embodiments, administering the glutamine comprises administering about 0.4-1.6 g/kg per day. In some embodiments, administering the glutamine comprises administering about 0.6-1.8 g/kg per day. In some embodiments, administering the glutamine comprises administering about 0.8-2.4 g/kg per day. In some embodiments, administering the glutamine comprises administering about 1.0-3.0 g/kg per day. These daily doses can be administered in one dose, or split between multiple doses; for example, 2, 3 or 4 doses.

In various embodiments, administering the glutamine comprises administering an amount of glutamine to achieve about 20-40 mM concentration in the subject. In various embodiments, administering the glutamine comprises administering an amount of glutamine to achieve about 10-20 mM concentration in the subject. In various embodiments, administering the glutamine comprises administering an amount of glutamine to achieve about 20-30 mM concentration in the subject. In various embodiments, administering the glutamine comprises administering an amount of glutamine to achieve about 30-40 mM concentration in the subject. In various embodiments, administering the glutamine comprises administering an amount of glutamine to achieve about 25-35 mM concentration in the subject. In various embodiments, administering the glutamine comprises administering an amount of glutamine to achieve about 10-40 mM concentration in the subject. In various embodiments, administering the glutamine comprises administering an amount of glutamine to achieve about 10-50 mM concentration in the subject. In various embodiments, administering the glutamine comprises administering an amount of glutamine to achieve about 10-60 mM concentration in the subject. In various embodiments, administering the glutamine comprises administering an amount of glutamine to achieve about 10-14 mM concentration in the subject. In various embodiments, administering the glutamine comprises administering an amount of glutamine to achieve about 15-19 mM concentration in the subject. In various embodiments, administering the glutamine comprises administering an amount of glutamine to achieve about 20-24 mM concentration in the subject. In various embodiments, administering the glutamine comprises administering an amount of glutamine to achieve about 25-29 mM concentration in the subject. In various embodiments, administering the glutamine comprises administering an amount of glutamine to achieve about 30-34 mM concentration in the subject. In various embodiments, administering the glutamine comprises administering an amount of glutamine to achieve about 35-39 mM concentration in the subject. In various embodiments, administering the glutamine comprises administering an amount of glutamine to achieve about 40-44 mM concentration in the subject. In various embodiments, administering the glutamine comprises administering an amount of glutamine to achieve about 45-49 mM concentration in the subject. In various embodiments, administering the glutamine comprises administering an amount of glutamine to achieve about 50-60 mM concentration in the subject. In various embodiments, administering the glutamine comprises administering an amount of glutamine to achieve about 60-75 mM concentration in the subject. In various embodiments, the subject has cancer. In other embodiments, the subject does not have cancer.

In various embodiments, administering the glutamine comprises administering an amount of glutamine to achieve at least 2, 5, 10, 15, 20, 25, 30, 35, 40, 45, or 50 times the physiologic concentration of glutamine in the subject. In various embodiments, administering the glutamine comprises administering an amount of glutamine to achieve about 2, 5, 10, 15, 20, 25, 30, 35, 40, 45, or 50 times the physiologic concentration of glutamine in the subject. In various embodiments, administering the glutamine comprises administering an amount of glutamine to achieve at least 2 times the physiologic concentration of glutamine in the subject. In various embodiments, administering the glutamine comprises administering an amount of glutamine to achieve at least 5 times the physiologic concentration of glutamine in the subject. In various embodiments, administering the glutamine comprises administering an amount of glutamine to achieve at least 10 times the physiologic concentration of glutamine in the subject. In various embodiments, administering the glutamine comprises administering an amount of glutamine to achieve at least 15 times the physiologic concentration of glutamine in the subject. In various embodiments, administering the glutamine comprises administering an amount of glutamine to achieve at least 20 times the physiologic concentration of glutamine in the subject. In various embodiments, administering the glutamine comprises administering an amount of glutamine to achieve at least 25 times the physiologic concentration of glutamine in the subject. In various embodiments, administering the glutamine comprises administering an amount of glutamine to achieve at least 30 times the physiologic concentration of glutamine in the subject. In various embodiments, administering the glutamine comprises administering an amount of glutamine to achieve at least 35 times the physiologic concentration of glutamine in the subject. In various embodiments, administering the glutamine comprises administering an amount of glutamine to achieve at least 40 times the physiologic concentration of glutamine in the subject. In various embodiments, administering the glutamine comprises administering an amount of glutamine to achieve at least 50 times the physiologic concentration of glutamine in the subject. In various embodiments, administering the glutamine comprises administering an amount of glutamine to achieve at least 55 times the physiologic concentration of glutamine in the subject. In various embodiments, administering the glutamine comprises administering an amount of glutamine to achieve at least 60 times the physiologic concentration of glutamine in the subject. In various embodiments wherein an “at least” amount of physiologic concentration of glutamine in the subject is to be achieved, there can be an upper limit set; for example, 100 times the physiologic concentration of glutamine in the subject, or 150 times the physiologic concentration of glutamine in the subject, or 200 times the physiologic concentration of glutamine in the subject. However, this is not required unless specifically specified; for example, in the claims.

In various embodiments, administering the glutamine comprises administering an amount of glutamine to achieve about 2 times the physiologic concentration of glutamine in the subject. In various embodiments, administering the glutamine comprises administering an amount of glutamine to achieve about 5 times the physiologic concentration of glutamine in the subject. In various embodiments, administering the glutamine comprises administering an amount of glutamine to achieve about 10 times the physiologic concentration of glutamine in the subject. In various embodiments, administering the glutamine comprises administering an amount of glutamine to achieve about 15 times the physiologic concentration of glutamine in the subject. In various embodiments, administering the glutamine comprises administering an amount of glutamine to achieve about 20 times the physiologic concentration of glutamine in the subject. In various embodiments, administering the glutamine comprises administering an amount of glutamine to achieve about 25 times the physiologic concentration of glutamine in the subject. In various embodiments, administering the glutamine comprises administering an amount of glutamine to achieve about 30 times the physiologic concentration of glutamine in the subject. In various embodiments, administering the glutamine comprises administering an amount of glutamine to achieve about 35 times the physiologic concentration of glutamine in the subject. In various embodiments, administering the glutamine comprises administering an amount of glutamine to achieve about 40 times the physiologic concentration of glutamine in the subject. In various embodiments, administering the glutamine comprises administering an amount of glutamine to achieve about 45 times the physiologic concentration of glutamine in the subject. In various embodiments, administering the glutamine comprises administering an amount of glutamine to achieve about 50 times the physiologic concentration of glutamine in the subject. In various embodiments, administering the glutamine comprises administering an amount of glutamine to achieve about 55 times the physiologic concentration of glutamine in the subject. In various embodiments, administering the glutamine comprises administering an amount of glutamine to achieve at about 60 times the physiologic concentration of glutamine in the subject.

In non-cancer subjects the typical physiologic concentration of glutamine in the blood is about 0.4-0.6 mM. Thus, in various embodiments, the above amounts can be relative to physiologic concentration of glutamine in non-cancer subjects. In cancer patients, for example, pancreatic cancer patients, the physiologic concentration of glutamine in the blood can be typically about 4-6 mM, although 10 mM has been observed. Thus, in various embodiments, the above amounts can be relative to physiologic concentration of glutamine in cancer patients; for example, in a pancreatic cancer patient.

In various embodiments, the first treatment cycle of the anticancer agent comprises administering the anticancer therapy on about days 1, 8, and 15. In various embodiments, the first treatment cycle is about 28 days. In various embodiments, the first treatment cycle of the anticancer agent comprises administering the anticancer treatment about once, twice or three times a week for about four weeks. In various embodiments, the first treatment cycle of the anticancer agent comprises administering the anticancer treatment about once, twice or three times a week for about three, four or five weeks. In various embodiments, the dosage of the anticancer therapy as described herein can be reduced as compared to the standard of care treatment dose. In various embodiments, it can be reduced by about 10%, 20%, 25%, 50%, or 75% as compared to the standard of care treatment dose. In various embodiments, it can be reduced by about 5-10% as compared to the standard of care treatment dose. In various embodiments, it can be reduced by about 10-20% as compared to the standard of care treatment dose. In various embodiments, it can be reduced by about 20-30% as compared to the standard of care treatment dose. In various embodiments, it can be reduced by about 30-40% as compared to the standard of care treatment dose. In various embodiments, it can be reduced by about 40-50% as compared to the standard of care treatment dose. In various embodiments, it can be reduced by about 50-60% as compared to the standard of care treatment dose. In various embodiments, it can be reduced by about 60-75% as compared to the standard of care treatment dose.

In various embodiments, the first treatment cycle of the immunotherapeutic agent comprises administering the immunotherapeutic therapy on about day 1, every 2, 3, 4, or 6 weeks. In various embodiments, the first treatment cycle is about 14, 21, 28 or 42 days. In various embodiments, the first treatment cycle of the immunotherapeutic agent comprises administering the immunotherapeutic treatment about once, twice or three times a week for about four weeks. In various embodiments, the first treatment cycle of the immunotherapeutic agent comprises administering the immunotherapeutic treatment about once, twice or three times a week for about two, three, four, five or six weeks. In various embodiments, the dosage of the immunotherapeutic treatment as described herein can be reduced as compared to the standard of care treatment dose. In various embodiments, it can be reduced by about 10%, 20%, 25%, 50%, or 75% as compared to the standard of care treatment dose. In various embodiments, it can be reduced by about 5-10% as compared to the standard of care treatment dose. In various embodiments, it can be reduced by about 10-20% as compared to the standard of care treatment dose. In various embodiments, it can be reduced by about 20-30% as compared to the standard of care treatment dose. In various embodiments, it can be reduced by about 30-40% as compared to the standard of care treatment dose. In various embodiments, it can be reduced by about 40-50% as compared to the standard of care treatment dose. In various embodiments, it can be reduced by about 50-60% as compared to the standard of care treatment dose. In various embodiments, it can be reduced by about 60-75% as compared to the standard of care treatment dose.

In various embodiments, the first treatment cycle of the radiation therapy comprises administering the radiation therapy on about days 1, 8, and 15. In various embodiments, the first treatment cycle is about 28 days. In various embodiments, the first treatment cycle of the radiation therapy comprises administering the radiation therapy about once, twice or three times a week for about four weeks. In various embodiments, the first treatment cycle of the radiation therapy comprises administering the radiation therapy about once, twice or three times a week for about one, two, three, four, five or six weeks. In various embodiments, the first treatment cycle of radiation therapy comprises administering radiation therapy about daily for 1, 2, 3, 4, 5, 6, 7, 8, 9, 10, 11, 12, 13, 14, 15, 16, 17, 18, 19, 21, 22, 23, 24, or 25 days. In various embodiments, the dosage of the radiation therapy as described herein can be reduced as compared to the standard of care treatment dose. In various embodiments, it can be reduced by about 10%, 20%, 25%, 50%, or 75% as compared to the standard of care treatment dose. In various embodiments, it can be reduced by about 5-10% as compared to the standard of care treatment dose. In various embodiments, it can be reduced by about 10-20% as compared to the standard of care treatment dose. In various embodiments, it can be reduced by about 20-30% as compared to the standard of care treatment dose. In various embodiments, it can be reduced by about 30-40% as compared to the standard of care treatment dose. In various embodiments, it can be reduced by about 40-50% as compared to the standard of care treatment dose. In various embodiments, it can be reduced by about 50-60% as compared to the standard of care treatment dose. In various embodiments, it can be reduced by about 60-75% as compared to the standard of care treatment dose.

In various embodiments, the solid tumor is pancreatic cancer. In various embodiments, the solid tumor is pancreatic cancer with KRas mutation. In various embodiments, the solid tumor is a tumor with KRas mutation. Additional examples of solid tumors treated by the present invention include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, head and neck cancer, and brain cancer; including, but not limited to, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary adenomas, neuroblastomas, and craniopharyngiomas. As the embodiments of the present invention provide for methods relating to solid tumors, it does not include lymphomas. In various embodiments, the cancer is not lymphoma.

In various embodiments, the solid tumor is not melanoma. Gabra et al. (Nat Commun. 2020 Jul 3;11(1):3326) showed that dietary glutamine supplementation can inhibit melanoma tumor growth. However, a KRas mutation is not a common driver mutation in melanoma. Thus, it would neither be reasonably predictable nor would there have been any reasonable expectation of success that glutamine supplementation would be beneficial for treating KRas mutated solid tumors.

In various embodiments, the anticancer agent is chemotherapy. Examples of chemotherapeutic agents include cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic akylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

In various embodiments, the chemotherapy is gemcitabine. In various embodiments the dose of gemcitabine is about 300, 400, 500, 600, 200, 800, 900, 1000, 1100 or 1200 mg/m².

In various embodiments, the chemotherapy is paclitaxel. In various embodiments, the chemotherapy is nab-paclitaxel. In various embodiments the dose of nab-paclitaxel is about 25, 50, 75, 100, 125, 150, or 175 mg/m².

TABLE 1
Exemplary Dosing Levels^{a}

| Dose | | | |
|---|---|---|---|
| Level | Gemcitabine Dosing | Nab-paclitaxel Dosing | L-glutamine Dosing^{b} |
| −2 | 600 mg/m² IV D1, 8, 15 | 75 mg/m² IV D1, 8, 15 | 0.1 g/kg oral BID |
| −1 | 800 mg/m² IV D1, 8, 15 | 100 mg/m² IV D1, 8, 15 | 0.1 g/kg oral BID |
| 0 | 1000 mg/m² IV D1, 8, 15 | 125 mg/m² IV D1, 8, 15 | 0.1 g/kg oral BID |
| 1 | 1000 mg/m² IV D1, 8, 15 | 125 mg/m² IV D1, 8, 15 | 0.2 g/kg oral BID |
| 2 | 1000 mg/m² IV D1, 8, 15 | 125 mg/m² IV D1, 8, 15 | 0.3 g/kg oral BID |

^{a}Every 28-day cycles,
^{b}rounded to nearest 10, 20, or, 30 g/day, upper limit of 30 g/day; started 1 week prior to chemotherapy

While Table 1 suggests an upper limit of 30 g/day for L-glutamine, the embodiments of the present invention are not limited to an upper limit of 30 g/day unless specifically indicated; for example, in the claims.

In various embodiments, glutamine is administered for about one week prior to administering the chemotherapeutic agent.

As such, in various embodiments, the method comprises administering about 0.1 g/kg of glutamine twice a day for about one week, then continuing to administer about 0.1 g/kg of glutamine twice a day, and administering about 600 mg/m² gemcitabine on Day 1, Day 8, and Day 15. In various embodiments, the method comprises administering about 0.1 g/kg of glutamine twice a day for about one week, then continuing to administer about 0.1 g/kg of glutamine twice a day, and administering about 800 mg/m² gemcitabine on Day 1, Day 8, and Day 15. In various embodiments, the method comprises administering about 0.1 g/kg of glutamine twice a day for about one week, then continuing to administer about 0.1 g/kg of glutamine twice a day, and administering about 1000 mg/m² gemcitabine on Day 1, Day 8, and Day 15. In various embodiments, the method comprises administering about 0.2 g/kg of glutamine twice a day for about one week, then continuing to administer about 0.2 g/kg of glutamine twice a day, and administering about 1000 mg/m² gemcitabine on Day 1, Day 8, and Day 15. In various embodiments, the method comprises administering about 0.3 g/kg of glutamine twice a day for about one week, then continuing to administer about 0.3 g/kg of glutamine twice a day, and administering about 1000 mg/m² gemcitabine on Day 1, Day 8, and Day 15.

In various embodiments, the method comprises administering about 0.1 g/kg of glutamine twice a day for about one week, then continuing to administer about 0.1 g/kg of glutamine twice a day, and administering about 75 mg/m² Nab-paclitaxel on Day 1, Day 8, and Day 15. In various embodiments, the method comprises administering about 0.1 g/kg of glutamine twice a day for about one week, then continuing to administer about 0.1 g/kg of glutamine twice a day, and administering about 100 mg/m² Nab-paclitaxel on Day 1, Day 8, and Day 15. In various embodiments, the method comprises administering about 0.1 g/kg of glutamine twice a day for about one week, then continuing to administer about 0.1 g/kg of glutamine twice a day, and administering about 125 mg/m² Nab-paclitaxel on Day 1, Day 8, and Day 15. In various embodiments, the method comprises administering about 0.2 g/kg of glutamine twice a day for about one week, then continuing to administer about 0.2 g/kg of glutamine twice a day, and administering about 125 mg/m² Nab-paclitaxel on Day 1, Day 8, and Day 15. In various embodiments, the method comprises administering about 0.3 g/kg of glutamine twice a day for about one week, then continuing to administer about 0.3 g/kg of glutamine twice a day, and administering about 125 mg/m² Nab-paclitaxel on Day 1, Day 8, and Day 15.

In various embodiments, the method comprises administering about 0.1 g/kg of glutamine twice a day for about one week, then continuing to administer about 0.1 g/kg of glutamine twice a day, and administering about 75 mg/m² paclitaxel on Day 1, Day 8, and Day 15. In various embodiments, the method comprises administering about 0.1 g/kg of glutamine twice a day for about one week, then continuing to administer about 0.1 g/kg of glutamine twice a day, and administering about 100 mg/m² paclitaxel on Day 1, Day 8, and Day 15. In various embodiments, the method comprises administering about 0.1 g/kg of glutamine twice a day for about one week, then continuing to administer about 0.1 g/kg of glutamine twice a day, and administering about 125 mg/m² paclitaxel on Day 1, Day 8, and Day 15. In various embodiments, the method comprises administering about 0.2 g/kg of glutamine twice a day for about one week, then continuing to administer about 0.2 g/kg of glutamine twice a day, and administering about 125 mg/m² paclitaxel on Day 1, Day 8, and Day 15. In various embodiments, the method comprises administering about 0.3 g/kg of glutamine twice a day for about one week, then continuing to administer about 0.3 g/kg of glutamine twice a day, and administering about 125 mg/m² paclitaxel on Day 1, Day 8, and Day 15.

In various embodiments, these amounts of glutamine are rounded to the nearest 5 g.

In various embodiments, the anticancer agent can be targeted therapies, such as an inhibitor for androgen signaling for prostate cancer. Examples of androgen signaling inhibitors include, but are not limited to abiraterone, enzalutamide, bicalutamide, apalutamide, and darolutamide. Additional examples of targeted therapies include but are not limited to EGFR (epidermal growth factor receptor) antagonist. Examples of EGFR inhibitors include, but are not limited to erlotinib, afatinib, and osimertinib.

In various embodiments, the glutamine can be administered with targeted cancer therapy. Glutamine can be co-administered with targeted therapies, such as an inhibitor for androgen signaling for prostate cancer. Examples of androgen signaling inhibitors include, but are not limited to abiraterone, enzalutamide, bicalutamide, apalutamide, and darolutamide. In various embodiments, the glutamine can be administered with targeted cancer therapy such as an EGFR (epidermal growth factor receptor) antagonist for lung cancer. Examples of EGFR inhibitors include, but are not limited to erlotinib, afatinib, and osimertinib.

In various embodiments of the invention, the therapeutically effective amounts of one or more anticancer agent or immunotherapeutic agent for use with the methods described herein may be in the range of 1-5 units/kg, 5-10 units/kg, 10-50 units/kg, 50-100 units/kg, 100-150 units/kg, 150-200 units/kg, 100-200 units/kg, 200-300 units/kg, 300-400 units/kg, or 400-500 units/kg. In some embodiments, the therapeutically effective amount of anticancer agent is about 25-50 units/kg, about 50-75 units/kg, about 75-100 units/kg or about 50 units/kg. In various embodiments, the therapeutically effective amount is about 50 units/kg. In various embodiments, the therapeutically effective amount is about 25-100 units/kg.

In some embodiments of the invention, the therapeutically effective amounts of the anticancer agent or immunotherapeutic agent can be in the range of about 1-5 μg/day, 5-10 μg/day, 10-15 μg/day, 15-20 μg/day, 10-20 μg/day, 20-30 μg/day, 30-40 μg/day, 40-50 μg/day, 50-60 μg/day, 60-70μg/day, 70-80 μg/day, 80-90 μg/day, 90-100 μg/day, 100-110 μg/day, 110-120 μg/day, 120-130 μg/day, 130-140 μg/day, 140-150 μg/day, 150-160 μg/day, 160-170 μg/day, 170-180 μg/day, 180-190 μg/day, 190-200 μg/day, 200-210 μg/day, 210-220 μg/day, 220-230 μg/day, 230-240 μg/day, 240-250 μg/day, 250-260 μg/day, 260-270 μg/day, 270-280 μg/day, 280-290 μg/day or 290-300 μg/day.

In some embodiments of the invention, the therapeutically effective amounts of the anticancer agent or immunotherapeutic agent can be in the range of about 10-50 μg/day, 50-100 μg/day, 100-150 μg/day, 150-200 μg/day, 100-200 μg/day, 200-300 μg/day, 300-400 μg/day, 400-500 μg/day, 500-600 μg/day, 600-700 μg/day, 700-800 μg/day, 800-900 μg/day, 900-1000 μg/day, 1000-1100 μg/day, 1100-1200 μg/day, 1200-1300 μg/day, 1300-1400 μg/day, 1400-1500 μg/day, 1500-1600 μg/day, 1600-1700 μg/day, 1700-1800 μg/day, 1800-1900 μg/day, 1900-2000 μg/day, 2000-2100 μg/day, 2100-2200 μg/day, 2200-2300 μg/day, 2300-2400 μg/day, 2400-2500 μg/day, 2500-2600 μg/day, 2600-2700 μg/day, 2700-2800 μg/day, 2800-2900 μg/day or 2900-3000 μg/day.

In some embodiments of the invention, the therapeutically effective amounts of one or more anticancer agent or immunotherapeutic agent can be in the range of about 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day.

In various embodiments, the effective amount of anticancer agent or immunotherapeutic agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/kg/day, or a combination thereof.

In various embodiments, the effective amount of the anticancer agent or immunotherapeutic agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/m²/day, or a combination thereof.

In various embodiments, the effective amount of the anticancer agent or immunotherapeutic agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m²/day, or a combination thereof.

In various embodiments, the effective amount of radiation therapy is any one or more of about 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, or 96-100 Gy. These effective amount may be given in one or more fractions; for example, 2, 3, 4, 5, 6, 7, 8, 9, 10, 11, 12, 13, 14, 15, 16, 17, 18, 19 or 20 fractions. In various embodiments the dose is about 0.5-1.0, 1.0-1.5, 1.5-2.0, or 2.0-2.5 Gy per fraction.

Here, “μg/kg/day” or “mg/kg/day” or “g/kg/day” refers to μg or mg or g agent per kg body weight of the subject per day, respectively, and “μg/m²/day” or “mg/m²/day” refers to μg or mg agent per m² body surface area of the subject per day, respectively.

In various embodiments, the methods are not intended to treat or prevent cachexia, not intended to reduce chemotherapy-induced gastrointestinal toxicity, not intended to treat chemotherapy-related neuropathy or not intended to stimulate the immune system.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral.

“Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

The present invention is also directed to a kit to treat a solid tumor. The kit is useful for practicing the inventive method of treating solid tumors. The kit is an assemblage of materials or components, including at least one of the inventive compositions. Thus, in some embodiments the kit contains a composition including glutamine, or glutamine and an anticancer agent such as gemcitabine or paclitaxel or Nab-paclitaxel as described herein.

The exact nature of the components configured in the inventive kit depends on its intended purpose. For example, some embodiments are configured for the purpose of treating solid tumors. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat the solid tumor with glutamine, or glutamine and an anticancer agent. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in anticancer treatment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

We accrue 16 patients with advanced or unresectable pancreatic cancer to this study based on an adaptive Bayesian design exploring 3 doses for gemcitabine, 600, 800, and 1000 mg/m² intravenous (IV) over 30 minutes on days 1, 8, and 15, 3 doses of nab-paclitaxel, 75, 100, and 125 mg/m² IV over 30 minutes on days 1, 8, and 15, and 3 doses of L-glutamine powder, 0.1, 0.2, and 0.3 g/kg oral twice daily (rounded to nearest 10, 20, or 30 g/day, maximum daily dose of 30 g/day), with L-glutamine starting 1 week prior to initiation of gemcitabine/nab-paclitaxel. A treatment cycle consists of 28 days with day 1 of cycle 1 beginning with the administration of gemcitabine/nab-paclitaxel.

We propose 5 dosing levels for the combination as proposed in Table 1. The primary endpoint will be the number of dose-limiting toxicities (DLTs) experienced within the first 4 weeks (1 cycle) of study treatment. A Bayesian adaptive design employing an extension of escalation with overdose control (EWOC) will be used to identify the maximum-tolerated dose (MTD), defined as the dose level such that the probability of DLT at the MTD is θ=0.33. The RP2D will be defined as the dose level closest to the median of the posterior distribution of the MTD. Starting subjects are enrolled to dose level 0. The computation of the dose to be administered to each subsequent patient and the estimate of the MTD will be carried out by a biostatistician using R and JAGS.

Dosing levels for gemcitabine and nab-paclitaxel are based on standard dose levels as recommended by the U.S. Food and Drug Administration (FDA)-labeled indication for this combination in metastatic PDAC. L-glutamine has been used widely as an oral supplement for various indications for decades with no evidence of major side effects. Proposed doses of oral L-glutamine is based on the only FDA-labeled indication for L-glutamine powder (Endari®) to date as per phase III trial data. In the preceding phase II study, L-glutamine was dosed based on body weight (0.3 g/kg/dose, rounded to nearest 10, 20, or 30 g/day) and adjusted in increments of 5 g with an upper limit of 30 g/day. Our proposal to start L-glutamine 1 week before the initiation of gemcitabine/nab-paclitaxel is consistent with recent phase III data supporting this approach. Across 47 prospective trials investigating enteral glutamine in over 1600 adult patients (including those treated with chemotherapy and radiation therapy), glutamine was well-tolerated without any definitive evidence of drug interactions or compromise in antitumor efficacy. We therefore anticipate that L-glutamine will be safe at full FDA-labeled doses in combination with gemcitabine/nab-paclitaxel. Rather, the dose-limiting feature of oral glutamine is in its administration as the solubility of glutamine is only 3.6% at 23° C. Consequently, doses of >15 g have historically required over 400 mL of fluid and constituted a significant burden to patients, who are often anorexic and would experience greater discomfort (nausea) with such continuous daily intake while on chemotherapy. The FDA-approved doses of L-glutamine now permit administration of the powder in 240 mL of fluid per dose, which translates to about 500 mL of fluid intake daily. In keeping with the constellation of prospective trial data whereby doses of oral glutamine have rarely, if at all, exceeded 30 g/day given that clinically-relevant larger volumes of water would be required to dissolve glutamine at higher doses, we have decided to cap the maximum daily dose of L-glutamine in this study to the FDA-recommended upper limit of 30 g/day.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of treating a solid tumor in a subject in need thereof, comprising:
administering a therapeutically effective amount of glutamine to the subject.

2. The method of claim 1, further comprising administering one or more treatment cycles of a therapeutically effective amount of anticancer agent, an immunotherapeutic agent, or radiation therapy to the subject.

3. The method of claim 2, comprising administering the glutamine about 3 days to 2 weeks prior to initiating the first treatment cycle of the anticancer agent, the immunotherapeutic agent, or radiation therapy.

4. The method of claim 2, comprising administering the glutamine about 1 week prior to initiating the first treatment cycle of the anticancer agent, the immunotherapeutic agent, or radiation therapy.

5. The method of claim 2, wherein the first treatment cycle of the anticancer agent or the immunotherapeutic agent comprises administering the anticancer therapy or the immunotherapeutic agent on about days 1, 8, and 15, or wherein the first treatment cycle of the radiation therapy comprises administering the radiation therapy once, twice or three times a week for about four weeks.

6. The method of claim 1, wherein the first treatment cycle is about 28 days.

7. The method of claim 1, wherein the glutamine is L-glutamine.

8. The method of claim 1, wherein the glutamine is D-glutamine.

9. The method of claim 1, wherein administering the glutamine comprises administering about 0.1 g/kg-1.2 g/kg of glutamine per day.

10. The method of claim 1, wherein administering the glutamine comprises administering about 0.2 g/kg-0.6 g/kg of glutamine per day.

11. The method any of claim 1, wherein administering the glutamine comprises administering glutamine twice daily.

12. The method of claim 1, wherein administering the glutamine comprises 10 g or 15 g of glutamine twice daily.

13. The method of claim 1, wherein administering the glutamine comprises administering an amount of glutamine to achieve about 10 mM to 50 mM concentration of glutamine in the subject.

14. The method of claim 1, wherein administering the glutamine comprises administering an amount of glutamine to achieve at about 5 to 50 times the physiologic concentration of glutamine in the subject.

15. The method of claim 1, wherein the solid tumor is a KRas mutated cancer.

16. The method of claim 1, wherein the solid tumor is pancreatic cancer.

17. The method of claim 1, wherein the solid tumor is KRas mutated pancreatic cancer

18. The method of claim 1, wherein the anticancer agent is chemotherapy.

19. The method of claim 18, wherein the chemotherapy is gemcitabine, nab-paclitaxel or paclitaxel.

20. (canceled)

21. A method of treating a solid tumor in a subject in need thereof, comprising:
administering a therapeutically effective amount of glutamine to the subject prior to administering one or more treatment cycles of a therapeutically effective amount of anticancer agent, an immunotherapeutic agent, or radiation therapy to the subject;
continuing to administer the therapeutically effective amount of glutamine to the subject; and
administering one or more treatment cycles of a therapeutically effective amount of anticancer agent, an immunotherapeutic agent, or radiation therapy to the subject.

22. The method of claim 21, wherein the glutamine is L-glutamine.

23. The method of claim 21, wherein the glutamine is D-glutamine.

24. The method of claim 21, comprising administering the therapeutically effective amount of glutamine to the subject about 3 days to 2 weeks prior to administering one or more treatment cycles of the therapeutically effective amount of anticancer agent, an immunotherapeutic agent, or radiation therapy to the subject.

25. The method of claim 21, comprising administering the therapeutically effective amount of glutamine to the subject about 1 week prior to administering one or more treatment cycles of the therapeutically effective amount of anticancer agent, an immunotherapeutic agent, or radiation therapy to the subject.

26. The method of claim 21, wherein the therapeutically effective amount of glutamine is about 0.1g/kg-1.2 g/kg per day.

27. The method of claim 21, wherein the therapeutically effective amount of glutamine is about 0.2g/kg-0.6 g/kg per day.

28. The method of claim 21,
wherein the one or more treatment cycles of the therapeutically effective amount of anticancer agent comprises administering one or more treatment cycles of gemcitabine, or
wherein the one or more treatment cycles of the therapeutically effective amount of anticancer agent comprises administering one or more treatment cycles of Nab-paclitaxel.

29. The method of claim 28, wherein the one or more treatment cycles of the therapeutically effective amount of anticancer agent comprises administering one or more treatment cycles of gemcitabine and wherein each treatment cycle of gemcitabine comprises about 600 mg/m²-1000 mg/m² of gemcitabine administered on day 1, day 8 and day 15.

30. (canceled)

31. The method of claim 28, wherein the one or more treatment cycles of the therapeutically effective amount of anticancer agent comprises administering one or more treatment cycles of Nab-paclitaxel and wherein each treatment cycle of Nab-paclitaxel comprises about 75 mg/m²-125 mg/m² of Nab-paclitaxel administered on day 1, day 8 and day 15.
